(19) United States
(12) Patent Application Publication
(10) Pub. No.: US 2026/0078083 A1
(43) Pub. Date: Mar. 19, 2026

(54) IMPROVED PROCESS FOR PREPARING P-NITROBENZOIC ACID-2-ETHYLHEXYL ESTER

(71) Applicant: BASF SE, Ludwigshafen am Rhein (DE)

(72) Inventors: Peter LEIDINGER, Ludwigshafen am Rhein (DE); Thomas BERG, Ludwigshafen am Rhein (DE); Helmut KRONEMAYER, Ludwigshafen am Rhein (DE); Joerg WERLE, Ludwigshafen am Rhein (DE)

(21) Appl. No.: 19/106,798

(22) PCT Filed: Aug. 28, 2023
(86) PCT No.: PCT/EP2023/073495

§ 371 Date: Feb. 26, 2025

(30) Foreign Application Priority Data
Aug. 29, 2022 (EP) 22192663.7

(51) Int. Cl. C07C 229/60; C07C 201/12; C07C 227/04; C07D 251/70

(52) U.S. Cl. CPC C07C 229/60; C07C 201/12; C07C 227/04; C07D 251/70

(57) ABSTRACT

The present invention relates to a process for preparing p-nitrobenzoic acid-2-ethyl hexylester comprising reacting p-nitrobenzoic acid, wherein an excess of 2-ethylhexanol acts as solvent and no additional solvent is added, or p-nitrobenzoic acid and 2-ethylhexanol are provided in equimolar amounts and no additional solvent is added, or 2-ethylhexanol is provided in substoichiometric amounts and no additional solvent is added. Further, the present invention relates to the p-nitrobenzoic acid-2-ethyl hexylester obtained by the process and having high purity and optimum product properties.

The present invention relates to a process for preparing p-nitrobenzoic acid-2-ethyl hexylester comprising reacting p-nitrobenzoic acid, wherein an excess of 2-ethylhexanol acts as solvent and no additional solvent is added, or p-nitrobenzoic acid and 2-ethylhexanol are provided in equimolar amounts and no additional solvent is added, or 2-ethylhexanol is provided in substoichiometric amounts and no additional solvent is added. Further, the present invention relates to the p-nitrobenzoic acid-2-ethyl hexylester obtained by the process and having high purity and optimum product properties.

Ethylhexyl triazone (Uvinul T 150, CAS number: 88122-99-0) is a known highly effective UVB filter. Due to its beneficial physical and chemical properties, it is widely used as ingredient in various cosmetic preparations such as anti-aging face care products and sunscreens. The exceptional photo-stability and absorptivity of ethylhexyl triazone leads to the requirement of only small concentrations to achieve a high sun protection factor. Further, the polarity of the structure provides good solubility in cosmetic oils as well as high affinity to the skin's keratin.

Therefore, an economically and environmentally optimal industrial process for the preparation of ethylhexyl triazone is highly desirable. Typically, ethylhexyl triazone is prepared in a 3-step process starting with the esterification of p-nitrobenzoic acid. The formed p-nitrobenzoic acid-2-ethylhexyl ester is then reduced to the p-aminobenzoic acid-2-ethylhexyl ester, which subsequently reacts with a cyanuric halide to the final product.

As regards the first step, EP 3 674 293 A1 discloses the preparation of p-nitrobenzoic acid-2-ethylhexyl ester by reacting p-nitrobenzoic acid with the alcohol in an organic solvent in the presence of a catalyst, such as sulfuric acid, p-toluene sulfonic acid, polyphosphoric acid, or thionyl chloride. However, the use of an organic solvent increases chemical and/or recycling costs and requires additional purification steps for organic solvent removal.

CN112321522 also discloses a process for the preparation of p-nitrobenzoic acid-2-ethylhexyl ester from p-nitrobenzoic acid and isooctyl alcohol. This process requires washing and liquid separation after the reaction is finished. Further, the process is carried out under reduced pressure conditions. However, a process requiring elaborate purification and/or reaction conditions is disadvantageous, particularly for industrial applications.

Therefore, it was an object of the present invention to provide an economically and environmentally improved process for the preparation of p-nitrobenzoic acid-2-ethylhexyl ester.

In this regard, it was one object to provide a process which ensures high product purity without the need for purification by washing and phase separation. Further, it was an object to provide a process which does not require costly reaction conditions, such as, e.g., reduced pressure conditions. In another embodiment, it was desired to provide a process which minimizes excess use of chemicals in the reaction. Further, it was an object to provide a process, which is technically and economically suitable for large-scale applications.

It has surprisingly been found that at least some of the above objects can be achieved by the subject matter of the present invention as described hereinafter and in the claims.

In one embodiment, the present invention relates to a process for preparing p-nitrobenzoic acid-2-ethyl hexylester comprising reacting p-nitrobenzoic acid with 2-ethylhexanol, wherein
(a) an excess of 2-ethylhexanol acts as solvent and no additional solvent is added; or
(b) p-nitrobenzoic acid and 2-ethylhexanol are provided in equimolar amounts and no additional solvent is added; or
(c) 2-ethylhexanol is provided in substoichiometric amounts and no additional solvent is added.

It has been surprisingly found by the inventors, that the process of the invention provides the desired product in high yield and purity without the need for additional solvents or complex product purification by washing, phase separation or distillation. Further, it has been found that the process does not require elaborate reaction conditions, such as, e.g., reduced pressure conditions, which also reduces the requirements on the reaction setup. Moreover, either in option (a) only a small excess of 2-ethylhexanol is used as solvent in the process or in option (b) and (c) no excess of 2-ethylhexanol is used representing a solvent-free process. This is particularly surprising considering the explosive tendencies of the nitro-compounds present in the reaction mixture. Using lower quantities of excess 2-ethylhexanol, however, has the advantage of less 2-ethylhexanol that has to be removed, wasted, or recycled after the reaction is finished. These advantages even more apply if no excess 2-ethylhexanol is used.

Thus, the present invention provides an environmentally and economically advantageous process for the preparation of p-nitrobenzoic acid-2-ethyl hexylester in high purity and yield, which is suitable for large scale industrial applications. This includes low reaction and purification complexity in terms of the required steps, chemicals, and conditions used.

In one embodiment of the invention, the reaction is performed in the presence of a catalyst, which is preferably selected from sulfuric acid, p-toluene sulfonic acid, methane sulfonic acid, polyphosphoric acid, thionyl chloride, and mixtures thereof.

In one embodiment of the invention, in option (a), the molar ratio of 2-ethylhexanol to p-nitrobenzoic acid is less than 1.5:1.

In one embodiment of the invention, the reaction is not performed under reduced pressure.

In one embodiment of the invention, the product is purified by gas/steam stripping.

In one embodiment of the invention, the amount of catalyst is below 10 mol %, preferably below 5 mol %.

In one embodiment of the invention, the reaction is performed at a temperature of up to 180° C. for a time period of up to 48 hours.

In one embodiment of the invention, the process further comprises preparing p-aminobenzoic acid-2-ethyl hexylester comprising reacting the p-nitrobenzoic acid-2-ethyl hexylester with hydrogen in the presence of a catalyst.

In one embodiment of the invention, the p-nitrobenzoic acid-2-ethyl hexylester is not purified by washing, distillation or phase separation prior to the reaction with hydrogen in the presence of a catalyst.

In one embodiment of the invention, the pH value is adjusted to a value in the range of from 4 to 10 prior to the reaction with hydrogen.

In one embodiment of the invention, after the reaction with hydrogen, phase separation is performed by adding a carbonate salt.

In one embodiment of the invention, the process further comprises preparing 2,4,6-trianilino-p-(carbo-2′-ethylhexyl-1′-oxy)-1,3,5-triazine having the following chemical formula

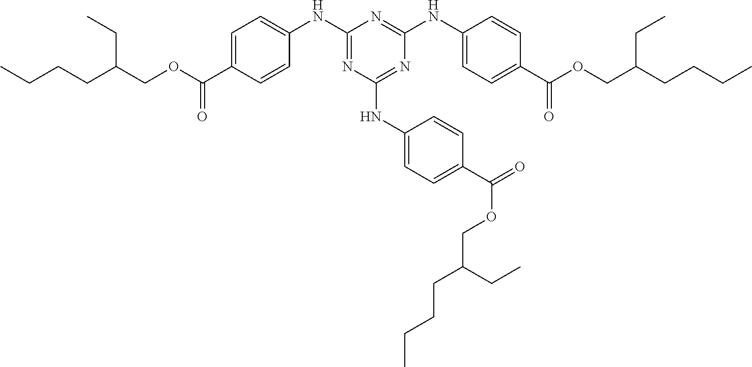 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

by reacting a cyanuric halide with the p-aminobenzoic acid-2-ethyl hexylester in a non-polar solvent.

In one embodiment, the present invention relates to p-nitrobenzoic acid-2-ethyl hexylester having a purity of at least 94% by weight.

In one embodiment, the present invention relates to p-nitrobenzoic acid-2-ethyl hexylester, which is obtained by the process of the present invention.

In one embodiment, the present invention relates to the p-nitrobenzoic acid-2-ethyl hexylester, wherein the amount of residual 2-ethylhexanol is less than 6% and/or the amount of residual p-nitrobenzoic acid is less than 2% and/or the amount of residual water is less than 1% and/or the amount of residual catalyst is less than 2%.

In one embodiment, the present invention relates to the p-nitrobenzoic acid-2-ethyl hexylester, which is in the form of a highly viscous fluid.

Preferred embodiments of the present invention can be found in the claims, the description and the examples. It is to be understood that the features mentioned above and those still to be illustrated below of the subject matter of the invention are preferred not only in the respective given combination but also in other combinations without leaving the scope of the invention.

In connection with the above embodiments of the present invention, the following definitions are provided.

In the context of the present invention p-nitrobenzoic acid-2-ethyl hexylester is obtained by a process comprising reacting p-nitrobenzoic acid with 2-ethylhexanol, wherein
(a) an excess of 2-ethylhexanol acts as solvent and no additional solvent is added; or
(b) p-nitrobenzoic acid and 2-ethylhexanol are provided in equimolar amounts and no additional solvent is added; or
(c) 2-ethylhexanol is provided in substoichiometric amounts and no additional solvent is added.

As used herein, “p-nitrobenzoic acid-2-ethyl hexylester” refers to para-nitrobenzoic acid-2-ethyl hexylester, which represents 4-nitrobenzoic acid-2-ethyl hexylester.

As used herein, “p-nitrobenzoic acid” refers to para-nitrobenzoic acid, which represents 4-nitrobenzoic acid (CAS number: 62-23-7). p-Nitrobenzoic acid is a commercially available, inexpensive compound and therefore especially suitable as a starting material in a large-scale process. 2-Ethylhexanol (104-76-7) is also commercially available and inexpensive and thus also suitable as a starting material in a large-scale process.

As used herein, the term “excess” refers to any amount providing a molar ratio exceeding a 1:1 molar ratio. In particular, an excess of 2-ethylhexanol as used herein refers to an amount of 2-ethylhexanol providing a molar ratio of 2-ethylhexanol relative to p-nitrobenzoic acid exceeding a 1:1 molar ratio.

As used herein, the term “equimolar amounts” refers to a 1:1 molar ratio. In particular, p-nitrobenzoic acid and 2-ethylhexanol provided in equimolar amounts refers to a 1:1 molar ratio of 2-ethylhexanol relative to p-nitrobenzoic acid.

As used herein, the term “substoichiometric amounts” refers to any amount providing a molar ratio below a 1:1 molar ratio. In particular, 2-ethylhexanol provided in substoichiometric amounts refers to an amount of 2-ethylhexanol providing a molar ratio of 2-ethylhexanol relative to p-nitrobenzoic acid below 1:1. Preferably, 2-ethylhexanol provided in substoichiometric amounts refers to an amount of 2-ethylhexanol providing a molar ratio of 2-ethylhexanol relative to p-nitrobenzoic acid between 0.5:1 and 1:1. If 2-ethylhexanol is provided in substoichiometric amounts, the yield is calculated relative to the applied molar amount of 2-ethylhexanol. In this case, the p-nitrobenzoic acid remaining after the reaction can be separated and re-used in the next reaction.

In some embodiments of the invention, the reaction is performed in the presence of a catalyst. The catalyst can be any Lewis acid or a Brønsted acid. Preferably the catalyst is selected from sulfuric acid, p-toluene sulfonic acid, methane sulfonic acid, polyphosphoric acid, thionyl chloride, or any other Lewis acid and mixtures thereof. As used herein, the catalyst can be used in its anhydrous form, as hydrate, as a solution, or in mixtures thereof. If the catalyst is used as a solution, it is preferably an aqueous solution. Further, if the catalyst is used as a solution, the percentage given together with the catalyst refers to the weight percentage of the catalyst and thus to the mass of the catalyst relative to the mass of the solution.

In some embodiments of the invention, the reaction is not performed under reduced pressure. The term “reduced pressure” as used herein refers to any pressure lower than atmospheric pressure.

In some embodiments of the invention, the product is purified by gas/steam stripping. Generally, stripping is a physical separation process which uses a vapor stream to remove components from a liquid sample. As used herein, gas/steam stripping is performed by feeding a reaction product into a vessel or reactor and stripping off the non-polar solvent and optionally side products by adding water or a polar solvent or water steam or a steam of a polar solvent or a gas or a mixture thereof to the vessel or reactor. Thus, gas/steam stripping according to the invention also includes flashing or flash, which describes the process of adding water or a polar solvent in the liquid phase from the top part of the vessel/reactor. Preferably, gas/steam stripping is performed by adding water steam, a steam of a polar solvent, a gas, or mixtures thereof from the top or bottom part of the vessel/reactor. More preferably, gas/steam stripping is performed by adding water steam, a steam of a polar solvent, a gas, or mixtures thereof from the bottom part of the vessel/reactor. The overhead stream that comprises the desorbed solvent and optionally side products of the reaction can be condensed and separated. As used herein, the term “steam” in connection with gas/steam stripping refers to water steam or steam of a polar solvent, in particular steam of an alcohol, preferably steam of ethanol. As used herein the term “gas” in connection with gas/steam stripping refers to inert gas that does not undergo chemical reactions, in particular nitrogen gas. As described above, the term gas/steam stripping according to the invention also includes flashing or flash with water or a polar solvent, preferably ethanol, in the liquid phase.

As used herein, the pressure indicated by the pressure unit “bar” refers to relative pressure or gauge pressure, which is zero-referenced against ambient air pressure, so it is equal to absolute pressure minus atmospheric pressure. Gauge pressure may also be indicated by the pressure unit “barg”. Only in case the pressure unit “bar(abs)” is used, the pressure refers to absolute pressure.

Preferred embodiments regarding the process of the invention are described hereinafter.

The following general considerations apply to the process.

In general, the reaction steps are performed in reaction vessels customary for such reactions, e.g., conventional stirred tank reactors. The reactions may be carried out in a continuous, semi-batch-wise or batch-wise manner. Preferably, the process of the invention is carried out in a batch-wise manner, wherein all reactants are provided in the reaction vessel before starting the reaction.

Details regarding the reaction pressure and temperature are provided below. If not indicated otherwise, the process steps are preferably carried out under atmospheric pressure. The end of the reaction can be monitored by methods known to a person skilled in the art, e.g., thin layer chromatography, GC, HPLC or NMR.

If not otherwise indicated, the reactants can in principle be contacted with one another in any desired sequence.

Furthermore, it is emphasized that the reaction may be performed on a laboratory scale and industrial scale.

In the following, preferred embodiments of the invention are provided. It is to be understood that the preferred embodiments of the invention are preferred alone or in combination with each other.

As indicated above, the present invention relates to a process for preparing p-nitrobenzoic acid-2-ethyl hexylester comprising reacting p-nitrobenzoic acid with 2-ethylhexanol, wherein
(a) an excess of 2-ethylhexanol acts as solvent and no additional solvent is added; or
(b) p-nitrobenzoic acid and 2-ethylhexanol are provided in equimolar amounts and no additional solvent is added; or
(c) 2-ethylhexanol is provided in substoichiometric amounts and no additional solvent is added.

Performing the reaction without an additional solvent avoids the presence of additional chemicals contaminating the reaction product and thus avoids more complex purification to remove said additional solvents after the reaction is finished. In one embodiment of the process of the invention, an excess of 2-ethylhexanol acts as solvent and no additional solvent is added. Using an excess of 2-ethylhexanol, i.e. the reactant itself, as solvent is further advantageous to increase conversion of the p-nitrobenzoic acid and thus the product yield. The removed excess of 2-ethylhexanol can subsequently be re-used in the next reaction. Moreover, if no product isolation is desired and the subsequent reaction can be performed in 2-ethylhexanol as solvent, the excess 2-ethylhexanol does not have to be removed after the reaction is finished. This applies, e.g., for the preparation of p-aminobenzoic acid-2-ethyl hexylester comprising reacting the p-nitrobenzoic acid-2-ethyl hexylester with hydrogen in the presence of a catalyst.

In another embodiment of the process of the invention, p-nitrobenzoic acid and 2-ethylhexanol are provided in equimolar amounts and no additional solvent is added. In this case, the reaction is performed without any solvent and can thus be considered solvent-free. Consequently, no solvent has to be removed, wasted or recycled after the reaction is finished. In another embodiment of the process of the invention, 2-ethylhexanol is provided in substoichiometric amounts and no additional solvent is added. Also in this case, the reaction is performed without any solvent and can thus be considered solvent-free. Consequently, no solvent has to be removed, wasted or recycled after the reaction is finished. Further, the excess of p-nitrobenzoic acid can be separated and applied in the next reaction. Considering the explosive tendencies of the nitro-compounds present in the reaction mixture, it is particularly surprising that the process of the present invention can be performed in a small excess of 2-ethylhexanol or even without any additional solvent.

In one embodiment of the process of the invention, the reaction is performed in the presence of a catalyst, which is preferably selected from an acid catalyst and even more preferably selected from sulfuric acid, p-toluene sulfonic acid, methane sulfonic acid, polyphosphoric acid, thionyl chloride and mixtures thereof. In one embodiment, the catalyst is p-toluene sulfonic acid. The catalyst can be used in its anhydrous form, as hydrate, as a solution, or in mixtures thereof. If the catalyst is used as a solution, it is preferably an aqueous solution. Further, if the catalyst is used as a solution, different weight percentages of the catalyst in the solution can be used. In one embodiment, the weight percentage of the catalyst in solution is above 50%. In another embodiment, the weight percentage of the catalyst in solution is above 60%. Regardless of whether the catalyst is used in its anhydrous form, as hydrate, as a solution, or in mixtures thereof, the catalyst can be applied in different amounts relative to the amount of p-nitrobenzoic acid used. If the catalyst is used as a solution, the amount of catalyst refers to the amount of catalyst in the solution.

In one embodiment of the invention, the amount of catalyst relative to the amount of p-nitrobenzoic acid is below 10 mol %, preferably below 5 mol %. In another embodiment, the amount of catalyst relative to the amount of p-nitrobenzoic acid is from 0.1 to 10 mol %, preferably 0.2 to 5 mol %. In another embodiment, the amount of catalyst relative to the amount of p-nitrobenzoic acid is from 0.5 to 2.5 mol %. In another embodiment, the amount of catalyst relative to the amount of p-nitrobenzoic acid is from 1.0 to 1.5 mol %. Reducing the amount of catalyst used, results in less catalyst waste or less catalyst recycling, which is advantageous for both preparation costs and environmental aspects. Further, the amounts of catalyst that must be removed after the reaction is finished during purification is lower.

As described above, in option (a) an excess of 2-ethylhexanol acts as solvent in the process of the invention for preparing p-nitrobenzoic acid-2-ethyl hexylester. This avoids the use of an additional solvent and thus its removal after the reaction is finished. In one embodiment of the invention, the molar ratio of 2-ethylhexanol relative to p-nitrobenzoic acid is less than 1.5:1. In another embodiment, the molar ratio of 2-ethylhexanol relative to p-nitrobenzoic acid is from 1.01:1 to 1.49:1. In another embodiment, the molar ratio of 2-ethylhexanol relative to p-nitrobenzoic acid is from 1.05:1 to 1.3:1. In another embodiment, the molar ratio of 2-ethylhexanol relative to p-nitrobenzoic acid is from 1.1:1 to 1.2:1. Using low amounts of excess 2-ethylhexanol as solvent is advantageous in terms of reducing cost and waste of chemicals as well as reducing solvent that has to be removed after the reaction is finished.

In one embodiment of the process of the invention, the reaction is not performed under reduced pressure. In another embodiment, the reaction is performed under atmospheric pressure. Not having to perform the reaction under reduced pressure provides the advantage of less process complexity and less requirements on the reaction setup. This can be associated with reduced process costs, in particular for large-scale industrial applications. In one embodiment, the reaction is performed under nitrogen atmosphere. In another embodiment, the reaction is performed under nitrogen atmosphere under atmospheric pressure.

In one embodiment of the process of the invention, the vessel or reactor is precharged with the catalyst under nitrogen atmosphere prior to the addition of 2-ethylhexanol and p-nitrobenzoic acid.

In one embodiment of the process of the invention, the reaction is performed upon heating. In one embodiment of the process of the invention, the reaction is performed under reflux. In one embodiment, the reaction is performed under reflux at a temperature of up to 180° C. for a time period of up to 48 hours. Performing the reaction at a temperature below 180° C. is advantageous in order to prevent decomposition of the product p-nitrobenzoic acid-2-ethyl hexyl ester at too high temperatures. Further, the upper limit of 180° C. has the advantage of using a temperature below the boiling point of 2-ethylhexanol. In another embodiment, the reaction is performed under reflux at a temperature of from 100° C. to 180° C. for a time period of from 1 h to 24 h. In another embodiment, the reaction is performed under reflux at a temperature of from 100° C. to 180° C. for a time period of from 2 h to 12 h. In another embodiment, the reaction is performed under reflux at a temperature of from 100° C. to 180° C. for a time period of from 4 h to 8 h. In another embodiment, the reaction is performed under reflux at a temperature of from 120° C. to 170° C. for a time period of from 1 h to 24 h. In another embodiment, the reaction is performed under reflux at a temperature of from 120° C. to 170° C. for a time period of from 2 h to 12 h. In another embodiment, the reaction is performed under reflux at a temperature of from 120° C. to 170° C. for a time period of from 4 h to 8 h. In another embodiment, the reaction is performed under reflux at a temperature of from 140° C. to 160° C. for a time period of from 1 h to 24 h. In another embodiment, the reaction is performed under reflux at a temperature of from 140° C. to 160° C. for a time period of from 2 h to 12 h. In another embodiment, the reaction is performed under reflux at a temperature of from 140° C. to 160° C. for a time period of from 4 h to 8 h.

In one embodiment of the process of the invention, the condensate of the refluxing reaction mixture is collected in a second vessel or reactor during the reaction. Thereby, water is separated during the reaction. In addition to water, the condensate may optionally comprise minor amounts of 2-ethylhexanol, catalyst and/or side-products. Thus, preferably the reaction is performed at a temperature below the boiling point of the catalyst if the condensate of the refluxing reaction mixture is collected in a second vessel or reactor during the reaction. The collected mixture of water and optionally 2-ethylhexanol, catalyst, and/or side products can be recycled. The aqueous phase can be separated, and the water purified. Alternatively, the catalyst-containing aqueous phase can be separated and re-used in following reactions as catalyst solution. Preferably, the catalyst-containing aqueous phase is purified before being re-used. Further, the 2-ethylhexanol can be separated and re-used in following reactions. Preferably, the 2-ethylhexanol is purified before being re-used.

In one embodiment of the process of the invention, the product is purified by steam/gas stripping after the reaction is finished. The resulting p-nitrobenzoic acid-2-ethyl hexyl ester is obtained in high yield and high purity. Thus, steam/gas stripping is advantageous in order to effectively remove water, the catalyst, optionally excess 2-ethylhexanol, optionally side products, and optionally remaining reactants from the desired p-nitrobenzoic acid-2-ethyl hexyl ester. Moreover, steam/gas stripping has the advantage of being applicable on a large industrial scale. Further, the mixture of water, the catalyst, optionally excess 2-ethylhexanol, optionally side products, and optionally remaining reactants separated by steam/gas stripping can be recycled. In particular, the catalyst-containing aqueous phase can be separated and re-used in following reactions as catalyst solution. Preferably, the catalyst-containing aqueous phase is purified before being re-used. Further, the 2-ethylhexanol can be separated and re-used in following reactions. Preferably, the 2-ethylhexanol is purified before being re-used.

In one embodiment, stripping is performed with water steam or a gas. In another embodiment, stripping is performed with a gas, which is selected from nitrogen or an alcohol, preferably ethanol in gaseous form. Preferably, the product is purified by stripping with nitrogen.

In one embodiment of the process of the invention, the temperature during steam/gas stripping is less than 180° C., preferably less than 170° C. Performing steam/gas stripping at a temperature below 180° C. is advantageous in order to prevent decomposition of the product p-nitrobenzoic acid-2-ethyl hexyl ester at too high temperatures. In another embodiment, the temperature during steam/gas stripping is from 100° C. to 180° C., preferably from 120° C. to 170° C. In another embodiment, the temperature during steam/gas stripping is from 140° C. to 160° C. In one embodiment, stripping is performed for a time period of up to 48 hours. In another embodiment, gas/steam stripping is performed for a time period of from 1 to 24 hours. In another embodiment, gas/steam stripping is performed for a time period of from 2 to 12 hours. In another embodiment, gas/steam stripping is performed for a time period of from 4 to 7 hours.

In one embodiment of the invention, the pressure of the gas/steam during gas/steam stripping is up to 100 bar, preferably up to 20 bar. In another embodiment, the pressure of the gas/steam during gas/steam stripping is from 0.1 bar to 20 bar. In another embodiment, the pressure of the gas/steam during gas/steam stripping is from 0.5 bar to 10 bar. In another embodiment, the pressure of the gas/steam during gas/steam stripping is from 1 bar to 6 bar. In one embodiment, the temperature during gas/steam stripping is from 120° C. to 170° C. and the pressure of the gas/steam during gas/steam stripping is from 0.1 bar to 20 bar. In one embodiment, the temperature during gas/steam stripping is from 120° C. to 170° C., and the pressure of the gas/steam during gas/steam stripping is from 0.5 bar to 10 bar. In another embodiment, the temperature during gas/steam stripping is from 140° C. to 160° C. and the pressure of the gas/steam during gas/steam stripping is from 1 bar to 6 bar. In one embodiment, the temperature during gas/steam stripping is from 140° C. to 160° C. and the pressure of the gas/steam during gas/steam stripping is from 0.1 bar to 20 bar. In another embodiment, the temperature during gas/steam stripping is from 140° C. to 160° C. and the pressure of the gas/steam during gas/steam stripping is from 0.5 bar to 10 bar. In another embodiment, the temperature during gas/steam stripping is from 140° C. to 160° C. and the pressure of the gas/steam during gas/steam stripping is from 1 bar to 6 bar.

In one embodiment of the process of the invention, the amount of gas/steam used for gas/steam stripping is at least 25% in weight relative to the crude product. In another embodiment, the amount of gas/steam used for gas/steam stripping is at least 50% in weight relative to the crude product.

In one embodiment, stripping is performed for a time period of up to 48 hours. In another embodiment, stripping is performed for a time period of from 1 to 24 hours. In another embodiment, stripping is performed for a time period of from 2 to 12 hours. In another embodiment, stripping is performed for a time period of from 4 to 7 hours. In another embodiment, the product is purified by stripping with nitrogen under atmospheric pressure at a temperature of from 150° C. to 170° C. for a time period of up to 48 hours. In another embodiment, the product is purified by stripping with nitrogen under atmospheric pressure at a temperature of from 150° C. to 170° C. for a time period of from 1 to 24 hours. In another embodiment, the product is purified by stripping with nitrogen under atmospheric pressure at a temperature of from 150° C. to 170° C. for a time period of from 2 to 12 hours. In another embodiment, the product is purified by stripping with nitrogen under atmospheric pressure at a temperature of from 150° C. to 170° C. for a time period of from 5 to 7 hours.

As indicated above, the process of the present invention provides a process for preparing p-nitrobenzoic acid-2-ethyl hexylester. p-Nitrobenzoic acid-2-ethyl hexylester is a common starting material for the preparation of p-aminobenzoic acid-2-ethyl hexylester. Thus, in one embodiment of the invention, the process further comprises preparing p-aminobenzoic acid-2-ethyl hexylester comprising reacting the p-nitrobenzoic acid-2-ethyl hexylester with hydrogen in the presence of a catalyst. As described above, high purity of the p-nitrobenzoic acid-2-ethyl hexylester can be obtained by the process of this invention without purification by washing, distillation or phase separation. Thus, in one embodiment of the invention, the raw p-nitrobenzoic acid-2-ethyl hexylester is not purified by washing, distillation or phase separation prior to the reaction with hydrogen in the presence of a catalyst for the preparation of p-aminobenzoic acid-2-ethyl hexylester. Therefore, the improved process for preparing p-nitrobenzoic acid-2-ethyl hexylester is also advantageous for the preparation of p-aminobenzoic acid-2-ethyl hexylester. Further, the purity of the p-aminobenzoic acid-2-ethyl hexylester can be increased by adjusting the pH prior to the reaction with hydrogen. Thus, in one embodiment of the invention, the pH value is adjusted to a value in the range of from 4 to 10 prior to the reaction with hydrogen. In another embodiment of the invention, after the reaction with hydrogen, phase separation is performed by adding a carbonate salt. Performing phase separation by adding the carbonate salt reduces the water content.

p-Aminobenzoic acid-2-ethyl hexylester can be reacted with a cyanuric halide to obtain the known highly effective UV absorber ethylhexyl triazone (Uvinul T 150, CAS number: 88122-99-0). Said ethylhexyl triazone represents 2,4,6-trianilino-p-(carbo-2′-ethylhexyl-1′-oxy)-1,3,5-triazine. Thus, in another embodiment, the process further comprises preparing 2,4,6-trianilino-p-(carbo-2′-ethylhexyl-1′-oxy)-1,3,5-triazine having the following chemical formula

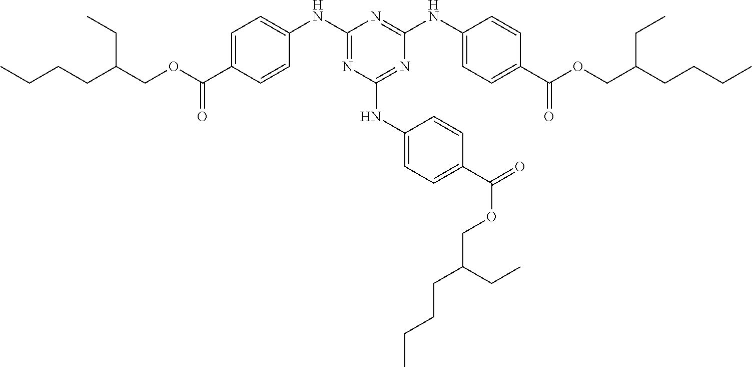 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

by reacting a cyanuric halide with the p-aminobenzoic acid-2-ethyl hexylester in a non-polar solvent.

Preferably, the cyanuric halide is reacted with the p-aminobenzoic acid-2-ethyl hexylester in a molar ratio of from 1:3 to 1:5. Reacting the cyanuric halide with the p-aminobenzoic acid-2-ethyl hexylester in a molar ratio of at least 1:3 allows for full conversion of the cyanuric halide since 3 equivalents of p-aminobenzoic acid-2-ethyl hexylester relative to 1 equivalent of cyanuric halide are needed to form ethylhexyl triazone. Therefore, the improved process for preparing p-nitrobenzoic acid-2-ethyl hexylester is also advantageous for the preparation of ethylhexyl triazone. Reducing complexity, waste, and cost of the preparation of p-nitrobenzoic acid-2-ethyl hexylester also reduces the overall complexity, waste, and cost of the preparation of ethylhexyl triazone. This is particularly advantageous considering the demand for ethylhexyl triazone due to its various applications as UV absorber.

As described above, high purity can be obtained by the process of this invention. Thus, in one embodiment, the present invention relates to a p-nitrobenzoic acid-2-ethyl hexylester having a purity of at least 94% by weight. In another embodiment, the present invention relates to a p-nitrobenzoic acid-2-ethyl hexylester having a purity of at least 95% by weight.

Further, in one embodiment, the present invention relates to a p-nitrobenzoic acid-2-ethyl hexylester, which is obtained by the process of this invention.

In another embodiment, the present invention relates to the p-nitrobenzoic acid-2-ethyl hexylester having a purity of at least 94% by weight, or at least 95% by weight, and/or obtained by the process of the invention, wherein the amount of residual 2-ethylhexanol is less than 6% and/or the amount of residual p-nitrobenzoic acid is less than 2% and/or the amount of residual water is less than 1% and/or the amount of residual catalyst is less than 2%.

In addition, the p-nitrobenzoic acid-2-ethyl hexylester having a purity of at least 94% by weight, or at least 95% by weight, and/or obtained by the process of the invention, and/or wherein the amount of residual 2-ethylhexanol is less than 6% and/or the amount of residual p-nitrobenzoic acid is less than 2% and/or the amount of residual water is less than 1% and/or the amount of residual catalyst is less than 2%, is in the form of a highly viscous fluid.

Thus, in another embodiment, the present invention relates to a p-nitrobenzoic acid-2-ethyl hexylester, which is in the form of a highly viscous fluid.

The present invention is further illustrated by the following example.

### EXAMPLES

The following abbreviations are used: p-NBAE: p-nitrobenzoic acid-2-ethyl hexyl ester, 2-EH: 2-ethylhexanol, p-TSA: p-toluene sulfonic acid, p-NBA: p-nitrobenzoic acid.

In the example, Karl Fischer Titration was performed on a Metrohm 890 Titrando with a 900 Touch Control and 803 TI Stand using a Metrohm 6.0338.100 Double PT-Wire Electrode.

In the example, GC analysis is performed on an Agilent Technologies 7890B using the following conditions and parameters:
Detector: FID
Solvent: Dichloromethane
Column: Optima 1701 by Macherey-Nagel (25 m, 0.53 mm ID, 1.00 μm film thickness)
Carrier Gas: Helium 25 mL/min
Injector temperature and detector temperature: 250° C.
Temperature program: 100° C. for 2 min, followed by 10° C./min until 150° C., followed by 50° C./min until 200° C. and keeping 200° C. for 2 min.

In the example, HPLC analysis is performed on an Agilent 1260 using the following conditions and parameters:
Detector: UV at 220 nm
Column: Spherisorb ODS-2 by Waters (10 μm, 250×4 mm)
Flow: 1.2 mL/min
Solvent: Acetonitrile with 0.1 vol % phosphoric acid (Solvent A) and deionized water with 0.1 vol % phosphoric acid (Solvent B)

### Gradient Program:

| Time | Solvent A | Solvent B |
|---|---|---|
| [min] | [vol %] | [vol %] |
| 0.00 | 50.0 | 50.0 |
| 3.00 | 50.0 | 50.0 |
| 3.01 | 80.0 | 20.0 |
| 12.00 | 80.0 | 20.0 |
| 12.01 | 50.0 | 50.0 |

### Example 1: Preparation of p-nitrobenzoic acid-2-ethyl hexyl ester

17.4 kg of 65% p-toluene sulfonic acid was added to a reactor A under inert nitrogen atmosphere. Afterwards, 495 kg of 2-ethylhexanol and 562.5 kg of solid p-nitrobenzoic acid was added to the same reactor. The esterification was started by heating the reactor under stirring to a temperature of 160° C. for a time period of 6 hours. The reaction was performed under reflux and the produced mixture of 2-ethylhexanol, water and side products was collected in a second reactor B. The 2-ethylhexanol in reactor B can be re-used after purification for following reactions. Water and side-products were pumped to the waste water treatment plant. The remaining 2-ethylhexanol, water and side-products in reactor A were stripped with nitrogen at a temperature of 160° C. for an additional time period of 6 hours to form the finished product of p-nitrobenzoic-2-ethyl hexylester. The purity observed with GC was 95.5%. Characterization of the final product was done using GC (p-NBAE, 2-EH, Other) and HPLC (p-NBA, p-TSA) and the water content was measured using Karl-Fischer titration. The results are shown in Table 1.

TABLE 1

| | Yield p- | p-NBAE | 2-EH | Other | p-NBA | p-TSA | H₂O |
|---|---|---|---|---|---|---|---|
| | NBAE kg | area % | area % | area % | weight % | weight % | weight % |
| Example 1 | 982.5 | 95.5 | 4.4 | 0.12 | 0.08 | 1.28 | 0.02 |

Other = non identified substances from GC peaks

1.-16. (canceled)

17. A process for preparing p-nitrobenzoic acid-2-ethyl hexylester comprising reacting p-nitrobenzoic acid with 2-ethylhexanol, wherein
(a) an excess of 2-ethylhexanol acts as solvent and no additional solvent is added; or
(b) p-nitrobenzoic acid and 2-ethylhexanol are provided in equimolar amounts and no additional solvent is added; or
(c) 2-ethylhexanol is provided in substoichiometric amounts and no additional solvent is added.

18. The process according to claim 17, wherein the reaction is performed in the presence of a catalyst.

19. The process according to claim 18, wherein the catalyst is selected from sulfuric acid, p-toluene sulfonic acid, methane sulfonic acid, polyphosphoric acid, thionyl chloride and mixtures thereof.

20. The process according to claim 17, wherein the molar ratio of 2-ethylhexanol to p-nitrobenzoic acid is less than 1.5:1.

21. The process according to claim 17, wherein the reaction is not performed under reduced pressure.

22. The process according to claim 17, wherein the product is purified by gas/steam stripping.

23. The process according to claim 18, wherein the amount of catalyst is below 10 mol %.

24. The process according to claim 17, wherein the reaction is performed at a temperature of up to 180° C. for a time period of up to 48 hours.

25. The process according to claim 17, wherein the process further comprises preparing p-aminobenzoic acid-2-ethyl hexylester comprising reacting the p-nitrobenzoic acid-2-ethyl hexylester with hydrogen in the presence of a catalyst.

26. The process according to claim 25, wherein the p-nitrobenzoic acid-2-ethyl hexylester is not purified by washing, distillation or phase separation prior to the reaction with hydrogen in the presence of a catalyst.

27. The process according to claim 25, wherein the pH value is adjusted to a value in the range of from 4 to 10 prior to the reaction with hydrogen.

28. The process according to claim 25, wherein after the reaction with hydrogen, phase separation is performed by adding a carbonate salt.

29. The process according to claim 25, wherein the process further comprises preparing 2,4,6-trianilino-p-(carbo-2′-ethylhexyl-1′-oxy)-1,3,5-triazine having the following chemical formula 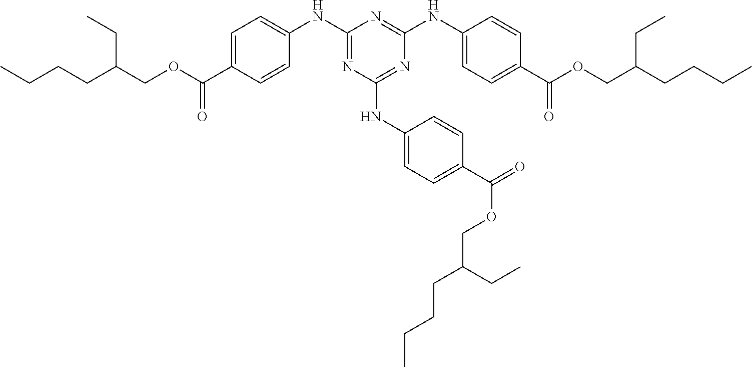 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]
by reacting a cyanuric halide with the p-aminobenzoic acid-2-ethyl hexylester in a non-polar solvent.

30. A P-Nitrobenzoic acid-2-ethyl hexylester having a purity of at least 94% by weight.

31. A P-Nitrobenzoic acid-2-ethyl hexylester having a purity of at least 94% by weight, which is obtained by the process of claim 17.

32. The p-nitrobenzoic acid-2-ethyl hexylester according to claim 30, wherein the amount of residual 2-ethylhexanol is less than 6% or the amount of residual p-nitrobenzoic acid is less than 2% or the amount of residual water is less than 1% or the amount of residual catalyst is less than 2%.

33. The p-Nitrobenzoic acid-2-ethyl hexylester according to claim 30, which is in the form of a highly viscous fluid.
